(19) United States
(12) Patent Application Publication
(10) Pub. No.: US 2015/0216784 A1
(43) Pub. Date: Aug. 6, 2015

(54) COSMETIC COMPOSITION CONTAINING FRAGMENTS OF BACTERIAL CELLULOSE FILM AND METHOD FOR MANUFACTURING THEREOF

(71) Applicant: NYMPHEAS INTERNATIONAL CORP., CHANGHUA COUNTY (TW)

(72) Inventors: YI-CHUAN LIN, CHANGHUA COUNTY (TW); YUH-CHYUN WEY, CHANGHUA COUNTY (TW); MEI-LING LEE, CHANGHUA COUNTY (TW); PIN-CHIH LIN, CHANGHUA COUNTY (TW)

(73) Assignee: NYMPHEAS INTERNATIONAL CORP., CHANGHUA COUNTY (TW)

(21) Appl. No.: 14/614,855

(22) Filed: Feb. 5, 2015

(30) Foreign Application Priority Data
Feb. 6, 2014 (TW) 103103888

(51) Int. Cl. A61K 8/73; A61Q 1/12; A61Q 1/14; A61Q 19/10; A61Q 5/02; A61Q 5/12; A61Q 13/00; A61Q 17/04; A61Q 1/06; A61Q 3/02

(52) U.S. Cl. CPC A61K 8/731; A61Q 1/06; A61Q 1/12; A61Q 1/14; A61Q 3/02; A61Q 5/02; A61Q 5/12; A61Q 13/00; A61Q 17/04; A61Q 19/10; A61K 2800/522; A61K 2800/805

(57) ABSTRACT

A cosmetic composition containing fragments of bacterial cellulose film is provided. The addition of the fragments of bacterial cellulose film in a cosmetic composition not only improves the transdermal transmission of active ingredients contained in the cosmetic composition but also provides the functions of skin moisturization, skin exfoliation and sebum absorption. Various applications of bacterial cellulose film are thus provided.

### CROSS REFERENCE TO RELATED APPLICATIONS

This application claims the priority of Taiwan Patent Application No. 103103888, filed Feb. 6, 2014 and entitled “COSMETIC COMPOSITION CONTAINING BACTERIAL CELLULOSE FILM FRAGMENTS AND METHOD FOR MANUFACTURING THEREOF”.

### TECHNICAL FIELD

The present invention relates to a cosmetic composition containing fragments of bacterial cellulose film and a method for manufacturing thereof.

### BACKGROUND

Bacterial cellulose (also named “BC” in the following), also named microbial cellulose or biocellulose, refers to cellulose produced by microorganisms. BC has a chemical structure identical to that in the plant but has a fiber diameter of approximately 2˜100 nm, about 1/100th of that in the plant. BC is formed by a linkage of β-1,4-glucosidic bonds between carbon atoms and shows high purity and water content in addition to excellent ductility and mechanical strength.

In general, bacterial cellulose is produced primarily in two ways: one is an aerobic fermentation of microorganisms under agitation, and the other is a static fermentation. The former has oxygen added so that the production velocity and yields are relatively high. However, the cellulose produced is tangled due to agitation and thus it is unable to form a membrane or film and show a high water content in this form. While, static fermentation makes bacteria produce cellulose in layers, a nonwoven reticular structure is formed on the surface of the culture medium. The membrane or film produced in this way contains extremely high water content.

BC has been broadly applied in biomedical materials, electronic materials, acoustics, cosmetics, food and the like. In the field of biomedicine, BC has been considered one of the best skin substitutes and has been commercialized as a dressing for burn wounds. It has been known that, BC is able to prevent the wounds from becoming infected, reduce pain, and absorb secretions from the wounds so as to enhance recovery. In addition, BC does not adhere to tissues or cells, which can prevent secondary damage to the tissues or cells. Moreover, BC possesses excellent biocompatibility and biosafety.

In the cosmetics industry, BC film are broadly used on nail care, skin care, lip care, long-lasting perfume design, patches for eye bags or weight loss, and the like. Because BC has a high water content and good gas permeability with no flowing fluids, it is suitable as a substrate for carrying cosmetically active ingredients and providing enhanced transdermal absorption.

### SUMMARY

A detailed description is given in the following embodiments with reference to the accompanying drawings.

An aspect of the invention provides a cosmetic formulation containing fragments of bacterial cellulose film. By a specific way of pulverization, the fragments of BC film, in the case of being a substrate of cosmetics, not only have the merits of small size for various application but also have properties of BC film to enhance the absorption of cosmetically active ingredients therein without destroying the original texture of the cosmetics.

Another aspect of the invention provides a cosmetic composition comprising fragments of BC film and a cosmetic additive, in which the fragments of BC film is 0.05˜1.0% by weight relative to the weight of the cosmetic composition.

Another aspect of the invention provides a method for manufacturing a cosmetic composition, comprising the following steps:

mixing a bacterial cellulose film with water for a first homogeneous pulverization to form a first solution, wherein the first solution contains 0.05˜1.0% by weight of the pulverized bacterial cellulose film, relative to the first solution;

diluting the first solution with water;

adding a surfactant or thickener to the diluted first solution for a second homogeneous pulverization to form a second solution; and

taking the second solution as a substrate, adding a cosmetically active ingredient and/or cosmetic additive into the second solution;

wherein the second solution contains fragments of the bacterial cellulose film, and

the fragments of the bacterial cellulose film are 0.05˜1.0% by weight relative to the weight of the cosmetic composition.

### BRIEF DESCRIPTION OF THE DRAWINGS

The present invention can be more fully understood by reading the subsequent detailed description and examples with references made to the accompanying drawings, wherein:

FIG. 1 is a bar chart showing the effect of a solution containing fragments of BC film on skin exfoliation according to one embodiment of the invention;

FIG. 2 is a bar chart showing the effect of a cream containing fragments of BC film on skin moisture according to one embodiment of the invention;

FIG. 3 is a bar chart showing the effect of a jelly masque containing fragments of BC film on removal of sebum according to one embodiment of the invention; and

FIG. 4 is a bar chart showing the effect of a facial cleanser containing fragments of BC film on facial cleansing according to one embodiment of the invention.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are shown schematically in order to simplify the drawing.

According to the invention, the term “bacteria cellulose film” (also named “BC film” in the following) refers to a film consisting of bacterial cellulose. In general, BC film are produced by culturing Gluconacetobacter, in particular G. xylinum, in a medium containing a carbon source such as glucose or maltose, yeast extracts, Na₂HPO₄ and citrate acid. However, in the present invention, a BC film includes all bacterial cellulose film produced by microorganisms without specific limitation. In the BC film, the water content may reach to 90˜98% by weight.

The term “fragments of bacterial cellulose film” according to the invention refers to those obtained by pulverizing BC film, which may have various forms, such as granules, sheets, gels, pastes or the like, and are not restricted to one single form in the invention.

Specifically speaking, the fragments of BC film according to the invention are obtained by a two-stage pulverization of BC film. In the “two-stage pulverization”, the first stage is homogenous pulverization of a mixture of BC film and water to form a first solution; and the second stage is another homogenous pulverization of a mixture of the first solution and a surfactant or thickener to form a second solution. The second solution contains the fragments of BC film and is used as a substrate of cosmetics.

According to the invention, by using the fragments of BC film from the two-stage pulverization being a substrate in addition with cosmetically active ingredients and/or cosmetic additives, a cosmetic composition can be made for various uses and have the functions of BC film without destroying the original properties of the cosmetically active ingredients and/or cosmetic additives.

More specifically, when the BC film is pulverized, the size of the film is reduced and the total surface area is increased. The fragments of BC film, being a substrate of cosmetics, are able to uniformly disperse in the cosmetic composition and become sufficiently mixed with other ingredients and/or additives. Since BC film in nature are highly stable and not easily volatilized, the cosmetic composition containing fragments of BC film can more stably stay on the skin surface so as to elongate the time period during which the cosmetically active ingredient is in contact with the skin surface and enhance the effects of the cosmetically active ingredient in the cosmetic composition to the skin.

When the cosmetic composition is for skin care, the addition of the fragments of BC film can lengthen the time period of the cosmetically active ingredient staying on the skin surface and thus increase the transdermal penetration and transmission of the cosmetically active ingredient in the cosmetic composition.

When the cosmetic composition is for foundation, make-up or the like, the addition of the fragments of BC film can make the make-up stable on the face and thus shorten the number of touch-ups required and reduce the amount of make-up needed. In addition, because the fragments of BC film have a small size, they can naturally fill the pores or scars on the face and make the face color even.

When the cosmetic composition is for a facial cleanser, it can remove mostly dead epidermal cells on the outermost skin surface but not cause physical damages thereto when scrubbing the face because of the small size and soft texture of the fragments of BC film.

The amount of the fragments of the BC film according to the invention may be 0.05˜1.0% by weight, preferably 0.1˜0.2% by weight in the final product, relative to the total weight of the cosmetic composition. However, the amount of the fragments of the BC film can be adjusted appropriately if it is deemed necessary.

According to the invention, the surfactant or thickener used in the two-stage pulverization may use that are well-known in the art, such as Arabic gum, Tween 80, methyl cellulose or a combination thereof, but it is not limited thereto.

The cosmetically active ingredient used in the invention, without specific limitation, can be appropriately selected and added according to the application. For example, the cosmetically active ingredient may be a moisturizer, such as hyaluronic acid or salicylic acid; a whitening ingredient, such as ursolic acid or kojic acid; an anti-wrinkle ingredient, such as polypeptides; an exfoliator, such as micro-granules; growth factors; enzymes; or a combination thereof; or the like.

The form of the cosmetic composition according to the invention may be selected from the group consisting of a toner, gel, jelly masque, clay masque, lotion, cream, lipstick, foundation, pressed powder, loose powder, makeup cosmetics, makeup-removing oil, makeup removing lotion, facial cleanser, bodywash, shampoo, hair conditioner, suntan lotion, hand cream, nail polish and perfume, but it is not limited thereto.

The cosmetic composition according to the invention may further comprise a cosmetic additive if necessary, such as an emulsifier, permeation enhancer, softener, solvent, excipient, antioxidant, or a combination thereof.

The examples in the following paragraphs are for describing the invention in detail, but it should be noted that the scope of the invention will not be limited in the following examples.

### Example 1

### Production of BC Film

A seed culture medium was prepared by using 10˜30 g/L of glucose and 5˜10 g/L of a yeast extract powder. Glyconacetobacter xylinus was then inoculated to the sterilized seed culture medium and culture under aeration at 30° C. for 3˜7 days to obtain the seed culture. Another culture medium containing 10˜30 g/L of glucose, 5˜10 g/L of a yeast extract powder, 2˜10 g/L of Na₂HPO₄ and 1˜5 g/L of citrate acid (sterilized under 121° C. for 30 minutes) was prepared. The seed culture was then inoculated into the culture medium with an inoculation rate of 5˜10% (v/v). The strain was statically fermented in A4-sized plates at 26˜32 for 3˜10 days. The bacterial cellulose film was then moved out of the plate and washed with 0.1%˜5% of a NaOH aqueous solution and soaked in the solution overnight. The film was further washed with water 3˜4 times and then neutralized with acids. The film obtained was stored for the subsequent steps.

### Example 2

### Manufacturing of Fragments of BC Film

7.5 kg of BC film (water content: about 98% by weight) was added into a pulverizer (TBSP-330) with 7.5 kg water and pulverized at 2,000 rpm. The pulverization was performed for a 4-minute duration every 5 minutes, the process lasting for more than 30 min. Due to the pulverization, the BC film became fragments. The pulverized BC film were then poured out and sterilized for 30 min. After cooling down, an anti-bacterial agent (methyl paraben (0.15% by weight) and methyl isothiazolinone (0.01% by weight)) was added into the pulverized BC film to form the first solution. The first solution was stored for later use.

Before use, 200 g sterilized water was added to 50 g of the first solution at a ratio of 4:1 and 0.2 wt % of Tween 80 was then added thereto. The solution was then put in a homogenizer and homogeneously pulverized for 30˜60 min to form the second solution. The second solution contained the fragments of BC film. During the preparation of cosmetics, the second solution could be used as a substrate for the addition of cosmetically active ingredients and/or cosmetic additives.

### Example 3

### Exfoliating Effect

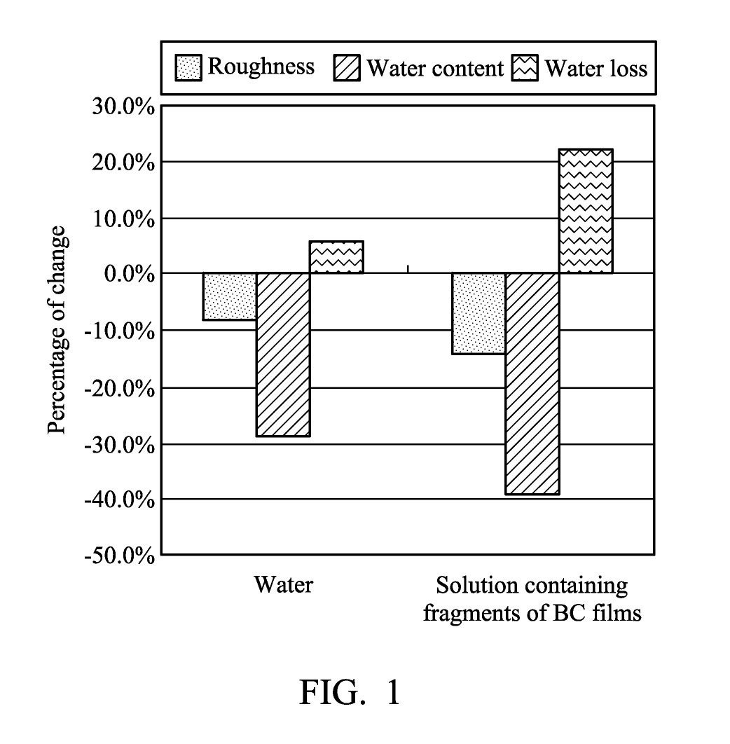

The second solution prepared in Example 2 was dropped on the human skin surface and rubbed for 30 sec. Water was used as a control. The rubbed skin surface was then examined to determine the roughness (CK Electronic), water content (Visioscan VC 98) and water loss rate (Tewameter TM 300). The results are shown in FIG. 1. Compared to the control (water-only treatment), the skin surface with the treatment of the second solution showed decreased roughness and water content with an increased water loss, indicating that the second solution containing the fragments of BC film had an effect on skin exfoliation.

### Example 4

### Transdermal Transmission Effect

The second solution with the fragments of BC film prepared in Example 2 was formulated according to the following Tables 1 and 2 to form a cream and jelly masque, respectively. As a control, water was used instead of the fragments of BC film. The effects of the fragments of BC film on the skin surfaces were estimated.

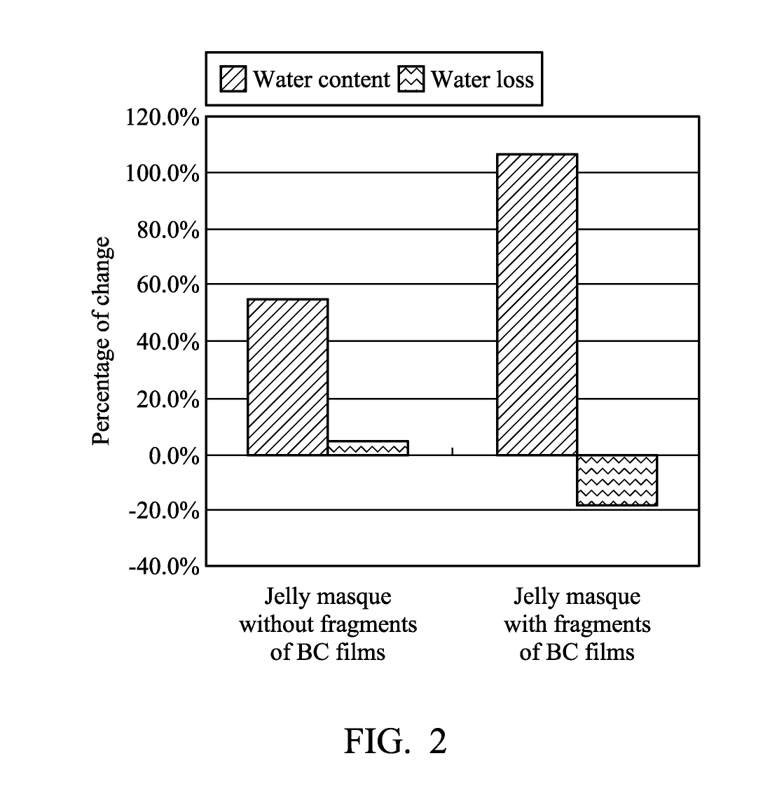

Thereafter, the jelly masque with the fragments of BC film was applied to human skin and left for 30 min. The results are shown in FIG. 2. According to FIG. 2, the skin surface with the treatment of the jelly masque containing fragments of BC film showed better effects on water loss and water content than the control group.

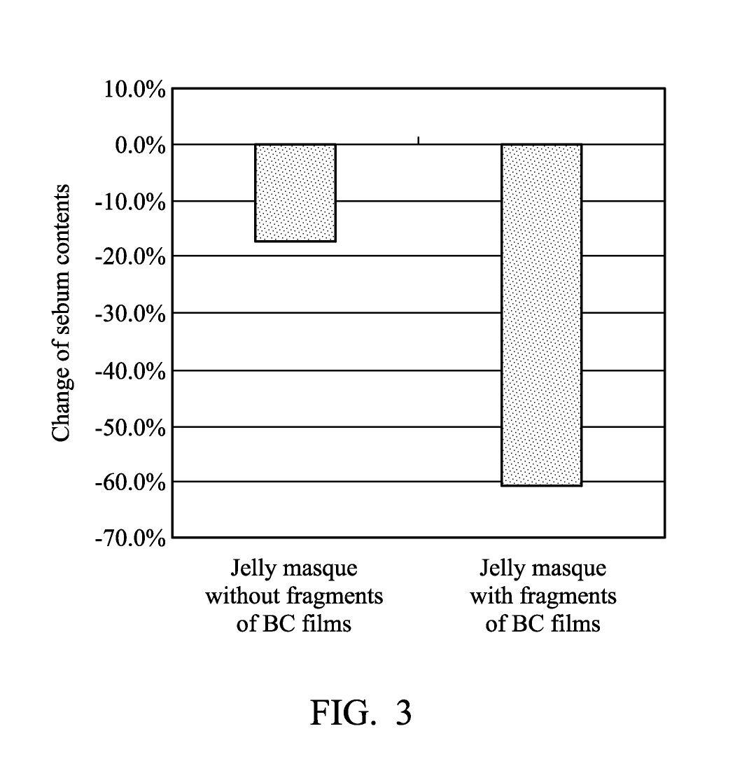

Subsequently, the jelly masque was washed out. The skin surface treated with the jelly masque was inspected for sebum content. The results are shown in FIG. 3. According to FIG. 3, the sebum on the skin surface treated with the jelly masque was largely decreased, indicating that the formulation containing fragments of BC film had the functions of reducing sebum content on the skin surface.

TABLE 1
Formulation of Cream

| | % by | | % by |
|---|---|---|---|
| ingredients | weight | ingredients | weight |
| Water | 81.99 | Hyaluronic acid | 5 |
| BC fragments | 0.5 | Panthenol | 0.1 |
| 1,3-butylene glycol | 3.0 | Xanthan gum | 0.2 |
| glycerin | 3.0 | Cucumber extract | 0.1 |
| isohexadecane | 2.0 | Tocopheryl acetate | 0.1 |
| Propylheptyl caprylate | 0.2 | Phenoxyethanol | 0.5 |
| Sodium polyacrylate | 3.0 | Methyl paraben | 0.01 |
| and paraffinum | | perfume | 0.2 |
| liquidum | | Vitamin B3 | 0.1 |

TABLE 2
Formulation of Jelly Masque

| | % by | | % by |
|---|---|---|---|
| ingredients | weight | ingredients | weight |
| Water | 79.19 | Carbomer | 15 |
| BC fragments | 1 | Xanthan gum | 1 |
| 1,3-butylene glycol | 3 | Cyclodextrin | 0.1 |
| Cucumber extract | 0.1 | Sodium lactate | 0.1 |
| Aloe extract | 0.1 | Methyl paraben | 0.01 |
| Sodium pyrrolidone | 0.1 | Perfume | 0.2 |
| carboxylic acid | | Tripleurospermum | 0.1 |
| | | Maritima extract | |

### Example 5

### Cleansing Effects

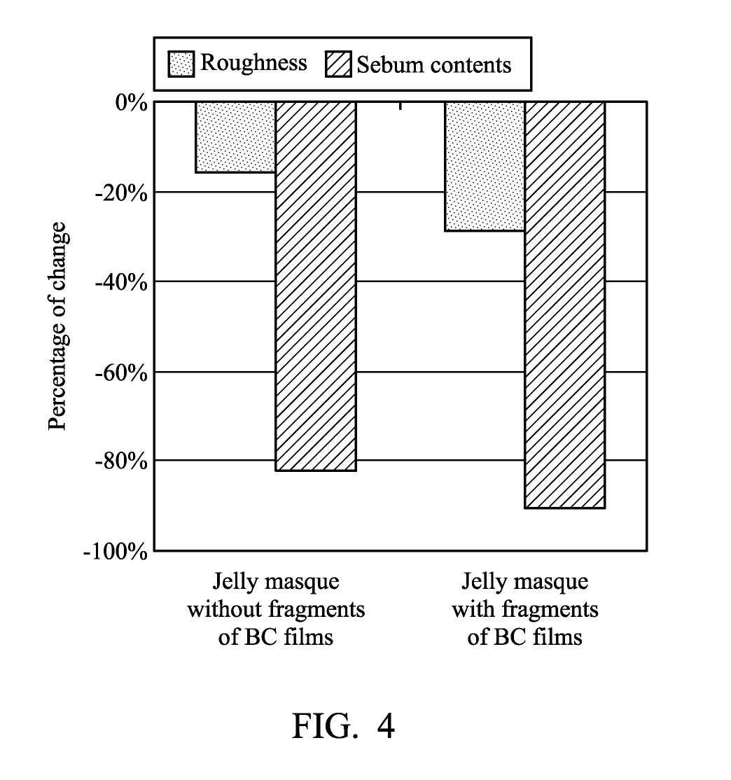

The second solution with fragments of BC film prepared in Example 2 was formulated according to the following Table 3 to form a facial cleanser. As a control, water was used instead of the fragments of BC film. The facial cleanser was then applied to human skin and rubbed for 1 min. The treated skin was examined to determine roughness and sebum content. The results are shown in FIG. 4. According to FIG. 4, the skin treated with facial cleanser containing fragments of BC film showed better effects on roughness and sebum content than the control group.

TABLE 3
Formulation of Facial Cleanser

| | % by | | % by |
|---|---|---|---|
| ingredients | weight | ingredients | weight |
| Water | 59.59 | 1,3-butylene glycol | 10 |
| BC fragments | 1 | Trehalose | 1 |
| Sodium lauroyl | 20 | Sodium pyrrolidone | 0.1 |
| glutamate | | carboxylic acid | |
| Sodium cocoyl | 3 | Dipotassium | 0.1 |
| glycinate | | glycyrrhizinate | |
| Glycerin | 5 | Methyl paraben | 0.01 |
| | | Perfume | 0.2 |

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A cosmetic composition, comprising fragments of a bacterial cellulose film and a cosmetic additive, wherein the fragment of a bacterial cellulose film is 0.05˜1.0% by weight relative to the weight of the cosmetic composition.

2. The cosmetic composition as claimed in claim 1, wherein the fragment of a bacterial cellulose film is produced by a two-stage pulverization of the bacterial cellulose film.

3. The cosmetic composition as claimed in claim 1, wherein the two-stage pulverization comprises steps of:
mixing a bacterial cellulose film with water for a first homogeneous pulverization to form a first solution, wherein the first solution contains 0.05˜1.0% by weight of the pulverized bacterial cellulose film, relative to the first solution;
diluting the first solution with water; and
adding a surfactant or thickener to the diluted first solution for a second homogeneous pulverization to form a second solution, wherein the second solution contains the fragments of the bacterial cellulose film.

4. The cosmetic composition as claimed in claim 3, wherein the surfactant or thickener comprises Arabic gum, Tween 80, methyl cellulose, or a combination thereof.

5. The cosmetic composition as claimed in claim 3, wherein water content in the bacterial cellulose film is 90˜98% by weight.

6. The cosmetic composition as claimed in claim 1, further comprising a cosmetically active ingredient.

7. The cosmetic composition as claimed in claim 6, wherein the cosmetically active ingredient comprises a moisturizer, whitening ingredient, anti-wrinkle ingredient, exfoliator, growth factor, enzyme, or the combination thereof.

8. The cosmetic composition as claimed in claim 1, wherein the cosmetic additive comprises an emulsifier, permeation enhancer, softener, solvent, excipient, antioxidant, or the combination thereof.

9. The cosmetic composition as claimed in claim 1, wherein the cosmetic composition is selected from the group consisting of a toner, gel, jelly masque, clay masque, lotion, cream, lipstick, foundation, pressed powder, loose powder, makeup cosmetics, makeup removing oil, makeup removing lotion, facial cleanser, bodywash, shampoo, hair conditioner, suntan lotion, hand cream, nail polish and perfume.

10. A method for manufacturing a cosmetic composition, comprising steps of:
mixing a bacterial cellulose film with water for a first homogeneous pulverization to form a first solution, wherein the first solution contains 0.05˜1.0% by weight of the pulverized bacterial cellulose film, relative to the first solution;
diluting the first solution with water;
adding a surfactant or thickener to the diluted first solution for a second homogeneous pulverization to form a second solution; and
taking the second solution as a substrate, adding a cosmetically active ingredient and/or cosmetic additive into the second solution;
wherein the second solution contains fragments of the bacterial cellulose film, and
the fragments of the bacterial cellulose film is 0.05˜1.0% by weight relative to the weight of the cosmetic composition.

11. The method as claimed in claim 10, wherein a water content in the bacterial cellulose film is 90˜98% by weight.

12. The method as claimed in claim 10, wherein the surfactant or thickener comprises Arabic gum, Tween 80, methyl cellulose, or a combination thereof.

13. The method as claimed in claim 10, wherein the cosmetically active ingredient comprises a moisturizer, whitening ingredient, anti-wrinkle ingredient, exfoliator, growth factor, enzyme, or the combination thereof.

14. The method as claimed in claim 10, wherein the cosmetic additive comprises an emulsifier, permeation enhancer, softener, solvent, excipient, antioxidant, or the combination thereof.

15. The method as claimed in claim 10, wherein the cosmetic composition is selected from the group consisting of a toner, gel, jelly masque, clay masque, lotion, cream, lipstick, foundation, pressed powder, loose powder, makeup cosmetics, makeup removing oil, makeup removing lotion, facial cleanser, bodywash, shampoo, hair conditioner, suntan lotion, hand cream, nail polish and perfume.

16. A cosmetic composition, produced by the method according to claim 10.
